(19) United States
(12) Patent Application Publication
(10) Pub. No.: US 2026/0248986 A1
(43) Pub. Date: Aug. 27, 2026

(54) RADIOPAQUE COATING FOR ENDOVASCULAR DEVICES

(71) Applicant: phenox GmbH, Bochum (DE)

(72) Inventors: Simon SCHLAGKAMP, Dorsten (DE); Jörg ASCHERFELD, Hattingen (DE); Volker TRÖSKEN, Witten (DE)

(73) Assignee: phenox GmbH, Bochum (DE)

(21) Appl. No.: 18/856,229

(22) PCT Filed: Apr. 13, 2023
(86) PCT No.: PCT/EP2023/059620

§ 371 Date: Oct. 11, 2024

(30) Foreign Application Priority Data
Apr. 13, 2022 (DE) 10 2022 109 090.6

(51) Int. Cl. A61L 31/08; A61L 31/18

(52) U.S. Cl. CPC A61L 31/088; A61L 31/18; A61L 2400/16; A61L 2420/02

(57) ABSTRACT

An endovascular device made of pseudoelastic alloys including a selective coating of radiopaque materials, the coating includes only regions or sections of the endovascular device which, in the radially maximally loaded state, have an elongation of less than 5% compared to the radially unloaded state of the device. Such a coating enables maximum X-ray visibility of the endovascular device with minimal impairment of the mechanical properties.

The invention relates to a radiopaque coating for endovascular devices, wherein the coating is selectively applied to the device in order to minimize the effect of the coating on the mechanical properties of the device.

Endovascular devices such as stents and other implants, filters, thrombectomy devices or flow diverters are usually very delicate objects. For successful placement and application in the vascular system, it is important to recognize their exact position already in the X-ray image during the intervention. However, due to their low mass and the materials used, many endovascular devices show little contrast in the X-ray image itself.

Accordingly, endovascular devices often include markers made of radiopaque materials such as gold or platinum, which allow the treating physician to better visualize the device during the intervention under X-ray imaging. However, such markers usually only mark certain areas of the device, such as the beginning or end of a stent.

In principle, however, it would be desirable for not only certain areas of the device to be better visualized in the X-ray image, but also for the entire device to be visible as far as possible. Visualizing only the end areas of a device provides little or no information as to whether, for example, it is also expanded as required in the area in between.

In order to achieve improved X-ray visibility of the device, the entire device can also be coated with a corresponding radiopaque material. However, the mechanical properties of the devices also change depending on the material and radiopaque material used.

Conventional materials such as steel or gold, for example, exhibit linear elasticity behavior, i.e. stresses and strains in the material increase proportionally under load until the elastic limit is reached. In this range, the loaded material also returns to its original shape after the load is removed. If the load is increased and the stress or strain exceeds the elastic limit, irreversible plastic deformation occurs.

Pseudoelastic alloys, also known as shape memory alloys, are characterized by a non-linear elasticity behaviour. They have the property of returning to their original shape even after considerable deformation when the load is removed.

Pseudo-elastic alloys such as Nitinol, NiTiCu, CuZn, CuZnAl or CuAINi can be stretched up to ten times more than conventional spring steels without being permanently deformed. The material undergoes a stress-strain hysteresis with a loading and unloading plateau.

In the case of endovascular devices whose basic structure consists of pseudoelastic alloys, a complete coating, which can also include a radiopaque material, has at least one decisive disadvantage. The applied coating increasingly restricts the pseudoelasticity of the material with increasing coating thickness. Important factors such as radial forces and set-up forces of the device or feed forces in the microcatheter are affected by this.

Accordingly, especially when coating endovascular devices made of shape memory alloys, a compromise must always be found between good visibility—through a higher coating thickness—and mechanical performance—through a lower coating thickness.

It is therefore the task of the invention to provide a coating and a coating method for endovascular devices made of shape memory alloys which do not have the disadvantages of known coatings and which enable improved X-ray visibility of the endovascular devices without unduly impairing the mechanical properties of the endovascular devices.

This problem is solved by the invention with the features of claims 1 and 16 and a method with the features of claims 6 and 9. Advantageous embodiments are the subject of the dependent claims. It should be pointed out that the features listed individually in the claims can also be combined with one another in any technologically meaningful way and thus demonstrate further embodiments of the invention.

Essential aim of the invention is to find a compromise between a coating that is as completely radiopaque as possible, that enables the device to be seen as fully as possible in the X-ray image and that at the same time has as little influence as possible on the mechanical properties of the device.

The essential idea of the invention is therefore based on coating only those regions of the device that are largely negligible for the mechanical properties of the device. Tests have shown that these are the regions of the device that experience only slight elongation when the device is used.

The mechanical properties of an endovascular device that are important in this context include, on the one hand, the radial resistive force (RRF), which counteracts external influences, i.e. the force exerted by the apparatus against external forces and the chronic outward force (COF) emanating from the device itself, which the device exerts on the surrounding tissue, such as the vessel wall, during and after release.

For a device made of a shape memory alloy, different considerations arise from the two quantities RRF and COF, since the diameter of the target vessel in which the device is inserted is usually smaller than the diameter of the device in its freely unfolded state.

When the initially compressed device is released from its transport system, for example the delivery catheter, it expands until it comes into contact with the vessel wall, which prevents its further expansion. Since the device attempts to return to its original shape, a low continuous force COF acts on the vessel wall at this point. The smaller the diameter of the device, i.e. the smaller the radial expansion of the device after release, the larger the COF acting on the vessel.

Another important quantity to mention is the RRF. RRF describes the force required, for example, to compress the device back to a smaller diameter by external pressures.

During storage or use of the device, individual regions are now stretched beyond the elastic limit of a conventional coating material such as gold, which can be applied to the device as a radiopaque marker. In contrast to the shape memory alloy of the device, this radiopaque layer of conventional coating material is thus permanently plastically deformed. As the layer thickness increases, this deformation impairs the pseudoelasiticity of the base structure of the device and consequently important performance characteristics of the device such as RRF and COF.

For example, the desired COF can no longer be achieved or a high RRF must be overcome in order to (re)insert the device into the transport system or the microcatheter.

In the selective coating according to the invention, only the regions or sections of the device that do not contribute or contribute only negligibly little to the mechanical performance, such as COF and RRF, are coated accordingly. These are regions that exhibit little mechanical stress and deformation during storage, insertion into the patient and use. In this way, X-ray visibility can be improved by high layer thicknesses on regions of the device that have little or no influence on mechanical performance. It is therefore not necessary to forego the advantages of the shape memory alloy.

Accordingly, the invention relates to an endovascular device made of pseudoelastic alloys, comprising a selective coating of radiopaque materials, wherein the coating comprises only regions or sections of the endovascular device which, in the radially maximally loaded state of the device, have an elongation in the corresponding region of less than 5%, preferably less than 3%, even more preferably less than 1.5% and in particular less than 1% compared to the device in the radially unloaded state.

In other words, the selective coating does not comprise regions and sections of the endovascular device which, in the radially maximally loaded state of the device, have an elongation of at least 5%, preferably of at least 3%, even more preferably of at least 1.5% and in particular of at least 1% compared to the device in the radially unloaded state.

It has been shown that by limiting the coating to the regions or sections that, in the radially maximally loaded state of the device, exhibit an elongation of less than 5%, preferably less than 3%, even more preferably less than 1.5% and in particular less than 1% as defined, a sufficient improvement in the X-ray visibility of the device can be achieved while at the same time maintaining the important mechanical properties.

Preferably, the coating further comprises only regions or sections of the endovascular device which, both in the radially maximally loaded state of the device and in the radially nominally loaded state, have an elongation in the corresponding region of less than 5%, preferably less than 3%, more preferably less than 1.5% and in particular less than 1% compared to the device in the radially unloaded state.

By definition, the elongation of the device is to be understood as the relative change in the length of a region of the device in a radially loaded state, for example the radially maximally or radially nominally loaded state, in relation to the radially unloaded state. The relative elongation of the device or of regions of the device in the maximally loaded state or in the maximally and nominally loaded state is essential for the idea of the invention.

As described, the selective coating does not include those regions or sections that have an elongation that reaches at least the defined limit value. Ideally, this means that the coating covers all regions that are below this limit value.

In practice, it has been shown that it is sufficient for the desired improvement in the mechanical properties of the device if the section of the device is not coated which includes the region of elongation which reaches at least the defined limit value. In the case of a stent, for example, the corresponding section of the respective strut is not coated.

When defining a section, it should be noted that it cannot be arbitrarily large and therefore includes an unreasonably large number of regions that are below the defined limit values for the elongation. Accordingly, a section should be defined as a part of the device that comprises precisely those regions that reach at least the defined limit value for the elongation. A region that reaches at least the defined limit value should also be called an elongation region.

Using the example of a stent-like device such as a stent, a thrombectomy device or a flow diverter, this means that at most the section of a strut should be defined as a section and accordingly not coated, that exactly encloses the elongation region, i.e. starts at the beginning of the elongation region and ends at the end of the elongation region, whereby the terms beginning and end refer to the course of the elongation region along the device, i.e. for example along a strut.

If several elongation regions are to be assigned to a section, for example because these elongation regions are located on different sides of a strut but directly in this section, at most that part of the strut should be defined as a section and not coated which exactly includes all these elongation regions, i.e. starts at the beginning of the first elongation region and ends at the end of the last elongation region, whereby the beginning and end can also be part of the same elongation region if this extends namely over all other elongation regions of the section.

An analogous definition can be used for other endovascular devices.

It is irrelevant for the invention how the endovascular device was manufactured from shape memory alloys. The device can, for example, be produced by cutting, in particular laser cutting, or by braiding, for example of wires, whereby stronger elongations are more likely to be expected with cut devices, for example laser-cut from a tube. However, other ways of manufacturing the device are also conceivable in principle, for example additive processes in which a structure is gradually built up by adding material. One such additive process is in particular 3D printing.

By definition, the radially maximally loaded state is the state of the device in which the device experiences its maximum radial compression. The device experiences the radially maximally loaded state, for example, in the delivery catheter before and during an intervention. If, for example, the device is intended for use with a delivery catheter with an inner diameter of 0.53 mm, the device experiences its maximum radially loaded state when radially compressed to a diameter of 0.53 mm. Radial compression to a diameter of 0.53 mm is the maximum radially loaded state for this device.

If the device is intended for use with delivery catheters of different internal diameters, the maximum radially loaded state for this device should be the state that the device assumes in the delivery catheter with the smallest internal diameter intended for this device.

If it is appropriate not to determine the radially maximally loaded state of the device as a function of a feed catheter, the radially maximally loaded state can alternatively be defined as the state in which the device reaches the limit of its pseudoelasticity and, under radial load, just does not yet have any regions that are permanently, i.e. no longer reversibly, deformed. Such a limit is generally reached with a device made of shape memory alloys if it has regions that exhibit an elongation of 10% compared to the radially unloaded state under radial compression.

In certain cases, such an alternative definition of the maximum radial load may be suitable for creating a further objective reference value.

The radially nominally loaded state is by definition the state of the device in which the device undergoes its nominal radial compression. The device experiences this state, for example, during use in accordance with the instructions, for example during implantation or other use in a vessel, for example as a vascular support, filter or for thrombectomy. If the device is accordingly intended for use in vessels with a diameter of 2 mm, the device experiences its nominal radially loaded state when radially compressed to a diameter of 2 mm. Radial compression to a diameter of 2 mm is the radially nominally loaded state for this device.

The radially loaded state of a device, be it the radially nominally loaded state or the radially maximally loaded state, should always be understood as a state with a uniformly distributed radial load on the device due to external force, such as is achieved in a blood vessel or a delivery catheter or, as a model, in the uniform compression of the device within a uniform straight tube.

By definition, the radially unloaded state is the state of the device in which the device does not experience any radial compression. The device experiences this state, for example, in the freely expanded state outside a vessel or a catheter without any external restrictions. Selective coating in the sense of the invention is achieved by all such coating methods which are capable of providing a ready-to-use end product with only a partial coating. It is irrelevant how the selective coating of the end product is achieved.

For example, the entire device can first be coated in the coating method and the coating can then be partially removed again mechanically, chemically or electrochemically. In the coating method, regions that are not to be coated can also be masked or covered in advance with various materials such as plastics, adhesives or covering lacquers. Corresponding masks can, for example, be removed again after coating or remain on the device. In addition, auxiliary elements can be attached inside and/or outside the device, which serve as a template during the coating method.

Last but not least, only the parts to be coated can be selectively coated. Various options are known to the skilled person that lead to the desired selective coating.

The pseudoelastic alloys or shape memory alloys can be nitinol (NiTi) or nickel-titanium-copper (NiTiCu) in particular. Other pseudoelastic alloys are known to those skilled in the art.

The material of the radiopaque coating can be selected from the group comprising platinum, palladium, platinum-iridium, tantalum, gold and tungsten. The radiopaque coating may also comprise a combination of two or more of these materials or alloys thereof. Accordingly, the radiopaque coating comprises at least one of the aforementioned materials. The person skilled in the art selects further radiopaque materials depending on the specific requirement and, if necessary, combines them with the aforementioned materials.

Accordingly, the invention comprises a method for selectively coating an endovascular device made of pseudoelastic alloys comprising the following steps:
(A) Providing an endovascular device made of pseudoelastic alloys in the radially unloaded state;
(D) Compressing the endovascular device to the radially maximally loaded state by radial loading of the device;
(E) Determine the regions of the endovascular device that have an elongation of at least 5% in the radially maximally loaded state compared to the radially unloaded expanded state;
(F) Coating the endovascular device except for the regions determined in step (E).

In a preferred embodiment, the method of selectively coating an endovascular device made of pseudoelastic alloys further comprises the following steps:
(B) Compressing the endovascular device to the radially nominally loaded state by radial loading of the device;
(C) Determine the regions of the endovascular device that have an elongation of at least 5% in the radially nominally loaded state compared to the device in the radially unloaded expanded state;

The steps (B) and (C) are usually carried out after step (A) and before step (D) according to their names.

In this variant of the method, the coating according to step (F) is carried out accordingly by recessing the regions determined in the steps (C) and (E).

The steps (B) and (C) can also be carried out instead of the steps (D) and (E). The coating according to step (F) is then carried out accordingly, leaving out the regions determined in step (C).

An alternative method for selectively coating an endovascular device made of pseudoelastic alloys comprises the following steps:
(A′) Providing an endovascular device made of pseudoelastic alloys in the radially unloaded state;
(D′) Compressing the endovascular device to the radially maximally loaded state by radial loading of the device;
(E′) Determining the regions of the endovascular device which, in the radially maximally loaded state, have an elongation of at least 5% compared with the radially unloaded expanded state, and then determining the sections of the endovascular device which comprise the corresponding regions of defined elongation;
(F′) Coating the endovascular device with the exception of the sections determined in step (E′).

In a preferred embodiment, the method of selectively coating an endovascular device made of pseudoelastic alloys further comprises the following steps:
(B′) Compressing the endovascular device to the radially nominally loaded state by radial loading of the device;
(C′) Determining the regions of the endovascular device which, in the radially nominally loaded state, have an elongation of at least 5% compared with the device in the radially unloaded expanded state, and then determining the sections of the endovascular device which comprise the corresponding regions of defined elongation;

The steps (B′) and (C′) are usually carried out after step (A′) and before step (D′) according to their names.

In this variant of the method, the coating according to step (F′) is carried out by recessing the sections determined in the steps (C′) and (E′) .

The steps (B′) and (C′) can also be carried out instead of the steps (D′) and (E′). The coating according to step (F′) is then carried out by recessing the sections determined in step (C′).

It is clear to the person skilled in the art that the steps (B) and (C) and the steps (D) and (E) can also be interchanged in sequence, i.e. after step (A), the steps (D) and (E) are carried out first and then the steps (B) and (C). The same applies to the steps (B′) and (C′) and the steps (D′) and (E′). These can also be interchanged in sequence, so that after step (A′), the steps (D′) and (E′) are carried out first and then the steps (B′) and (C′).

The elongation according to the steps (C) and (E) or according to the steps (C′) and (E′) can preferably be at least 3%, more preferably at least 1.5% and in particular at least 1% compared to the radially unloaded state.

The device can preferably be inserted into a straight tube with a corresponding internal diameter for compression to the radially nominally loaded state according to method step (B) or (B′). The inner diameter of the tube should correspond to the outer diameter of the device in the radially nominally loaded state. The tube is advantageously rigid and does not yield when the device expands. This allows comparable results to be obtained.

The device can be inserted into a straight tube with a corresponding internal diameter for compression to the radially maximally loaded state according to method step (D) or (D′). The inner diameter of the tube should correspond to the outer diameter of the device in the radially maximally loaded state. The tube is advantageously rigid and does not yield when the device expands. This allows comparable results to be obtained.

In principle, it is also conceivable that the method steps (A) to (E) or (A′) to (E′) are carried out in a computer simulation, for example using the finite element method (FEM), which models the real test setup described.

Accordingly, the invention also comprises an endovascular device with a coating obtainable by the inventive method.

The invention further comprises a combination comprising an endovascular device coated according to the invention and a delivery catheter for this device.

A device according to the invention with the selective coating according to the invention has the advantage over the prior art that the device is also almost completely visible in the X-ray image due to an almost complete coating, but without the coating impairing important mechanical properties such as RRF and COF to a relevant extent.

The effectiveness of the proposed selective coating is illustrated below using an example.

Example: In endovascular devices, such as stent systems, made of shape memory alloys, the COF decreases continuously with increasing expansion and a correspondingly larger stent diameter. In such devices made of shape-memory alloys which are completely coated with radiopaque materials such as gold, the COF deteriorates considerably, i.e. it decreases. This effect can be minimized by selectively coating the regions that do not contribute significantly to the COF.

In the present examples, a selective coating was applied to the regions or sections of the device made of a shape memory alloy which, according to the definition, showed an elongation of less than 1.5% in the radially maximally loaded or radially nominally loaded state.

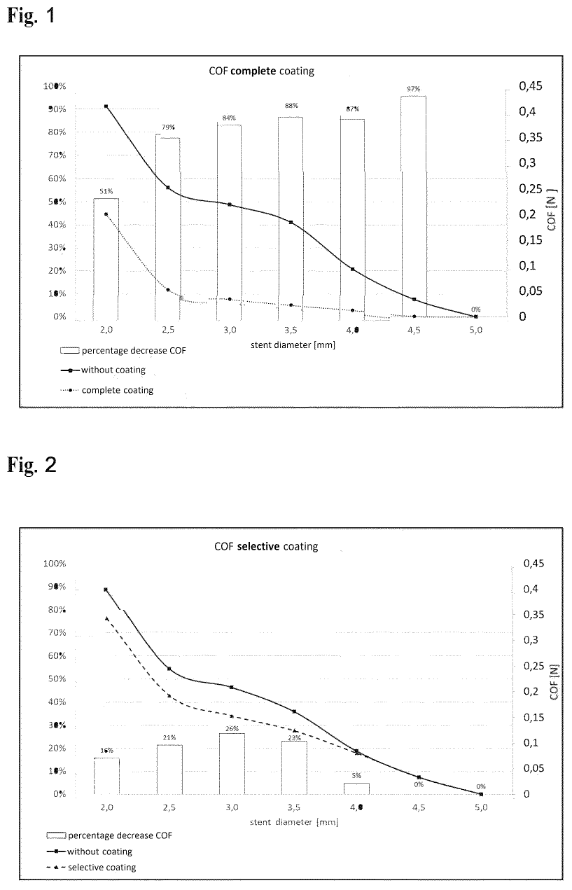

FIG. 1 shows in comparison the course of COF for a fully coated endovascular device made for a shape memory alloy (lower curve) and a fully uncoated endovascular device made of a shape memory alloy (upper curve), which is expanded from its radially nominally loaded state, in which the diameter of the device is 2 mm, to its radially unloaded state, in which the diameter of the device is 5 mm. It is easy to see that the COF of the fully coated device is significantly lower than that of the uncoated device, with a loss of between 51% and 97%.

FIG. 2 shows in comparison the course of COF for a selectively coated device made for a shape memory alloy according to the invention (lower curve) and a completely uncoated device made of a shape memory alloy (upper curve) which is expanded from its radially nominally loaded state, in which the diameter of the device is 2 mm, to its radially unloaded state, in which the diameter of the device is 5 mm. It is easy to see that the COF of the selectively coated device, with a maximum loss of COF of only 26%, is significantly lower than that of the uncoated device.

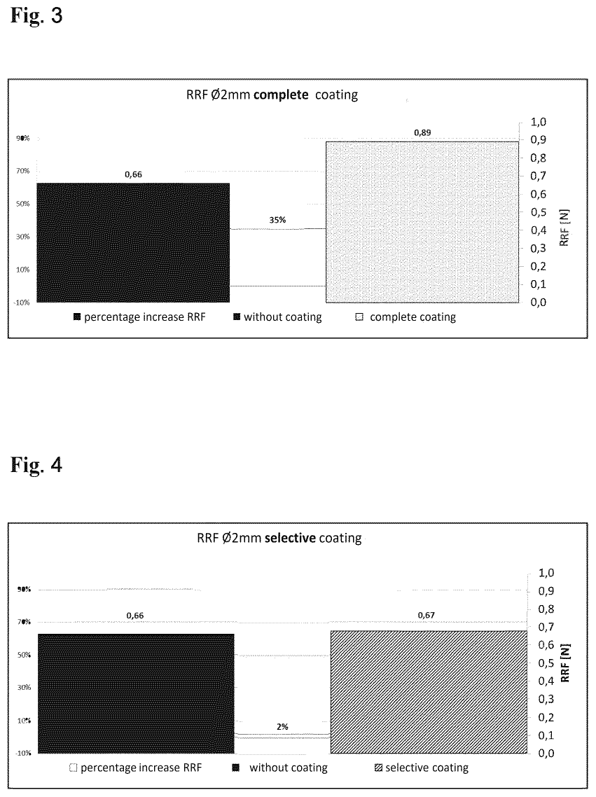

FIG. 3 shows in comparison the increase in RRF for a fully coated endovascular device made of a shape memory alloy (right bar) and an uncoated endovascular device made of a shape memory alloy (left bar), in each case in the radially nominally loaded state, which is at a diameter of 2 mm. It is easy to see that the RRF of the fully coated device, with a percentage increase of 35%, is significantly higher than that of the uncoated device.

FIG. 4 shows in comparison the increase in RRF for a selectively coated endovascular device made of a shape memory alloy (right bar) and an uncoated endovascular device made of a shape memory alloy (left bar), in each case in the radially nominally loaded state, which is at a diameter of 2 mm. It is easy to see that the RRF of the selectively coated device according to the invention, with a percentage increase of only 2%, is only slightly higher than that of the uncoated device.

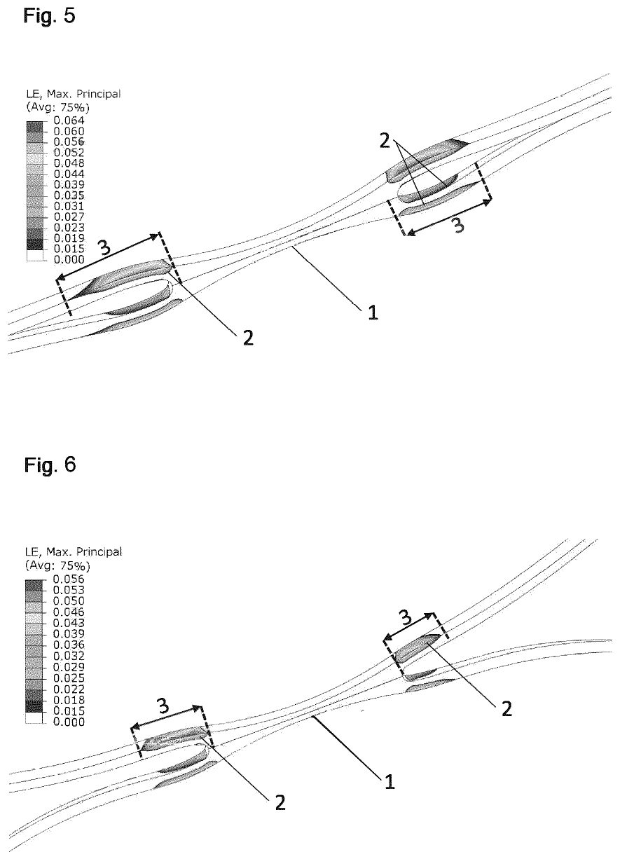

FIG. 5 shows the determination according to the invention of the regions of an endovascular device made of a shape memory alloy that are stressed by compression. In the present case, the data on the elongation of the regions 2 of the device or the struts 1 were determined in a computer simulation using the finite element method (FEM). The marked regions 2 represent the regions of the device or the struts 1 whose elongation during compression in the microcatheter, i.e. in the radially maximally loaded state, is at least 1.5% compared to the radially unloaded state. These regions 2 are omitted in a coating according to the invention.

Furthermore, FIG. 5 shows the sections 3 of the struts 1 derived from the regions 2, which can alternatively be omitted during selective coating.

FIG. 6 shows the determination according to the invention of the regions 2 or struts 1 of an endovascular device made of a shape memory alloy that are stressed by the compression. In the present case, the data on the elongation of the regions 2 or the struts 1 of the endovascular device were determined in a computer simulation using the finite element method (FEM). The marked regions 2 represent the regions 2 or the struts 1 of the endovascular device whose elongation during compression in the vessel, i.e. in the radially nominally loaded state, is at least 1.5% compared to the radially unloaded state. These regions 2 are omitted in a coating according to the invention.

Furthermore, FIG. 6 shows the sections 3 of the struts 1 derived from the regions 2, which can alternatively be omitted during selective coating.

The FIGS. 1 to 6 each refer to laser-cut endovascular devices.

1. An endovascular device made of pseudoelastic alloys comprising a selective coating of radiopaque materials, wherein the coating comprises only regions or sections of the endovascular device which, in the radially maximally loaded state of the device, have an elongation of less than 5% compared with the radially unloaded state of the device.

2. An endovascular device made of pseudoelastic alloys comprising a selective coating of radiopaque materials according to claim 1, wherein the coating comprises only regions or sections of the endovascular device which, both in the radially maximally loaded state and in the radially nominally loaded state of the device, have an elongation of less than 5% compared with the radially unloaded state of the device.

3. An endovascular device according to claim 1, wherein the elongation in the radially maximally or radially nominally loaded state is below 3%, preferably below 1.5% and in particular below 1% compared to the radially unloaded state.

4. An endovascular device according to claim 1, wherein the coating comprises at least one radiopaque material selected from the group consisting of platinum, palladium, platinum-iridium, tantalum, gold and tungsten.

5. An endovascular device according to claim 2, wherein the pseudoelastic material comprises NiTi or NiTiCu.

6. A method of selectively coating an endovascular device made of pseudoelastic alloys comprising the following steps:
(A) providing an endovascular device made of pseudoelastic alloys in the radially unloaded state;
(D) compressing the endovascular device to the radially maximally loaded state by radial loading of the device;
(E) determine the regions of the endovascular device that have an elongation of at least 5% in the radially maximally loaded state compared to the radially unloaded expanded state; and
(F) coating the endovascular device except for the regions determined in step (E).

7. A method of selectively coating an endovascular device made of pseudoelastic alloys according to claim 6, further comprising the steps of:
(B) compressing the endovascular device to the radially nominally loaded state by radial loading of the device; and
(C) determine the regions of the endovascular device that have an elongation of at least 5% in the radially nominally loaded state compared to the device in the radially unloaded expanded state;
wherein the coating according to step (F) is carried out by recessing the regions determined in steps (C) and (E).

8. A method for selectively coating an endovascular device made of pseudoelastic alloys according to claim 7, wherein the steps (D) and (E) are carried out after step (A) and before the steps (B) and (C).

9. A method of selectively coating an endovascular device made of pseudoelastic alloys comprising the following steps:
(A′) providing an endovascular device made of pseudoelastic alloys in the radially unloaded state;
(D′) compressing the endovascular device to the radially maximally loaded state by radial loading of the device;
(E′) determining the regions of the endovascular device which, in the radially maximally loaded state, have an elongation of at least 5% compared with the radially unloaded expanded state, and then determining the sections of the endovascular device which comprise the corresponding regions of defined elongation; and
(F′) coating the endovascular device with the exception of the sections determined in step (E′).

10. A method of selectively coating an endovascular device made of pseudoelastic alloys according to claim 9, further comprising the steps of:
(B′) compressing the endovascular device to the radially nominally loaded state by radial loading of the device; and
(C′) determining the regions of the endovascular device which, in the radially nominally loaded state, have a defined elongation of at least 5% compared with the device in the radially unloaded expanded state, and then determining the sections of the endovascular device which comprise the corresponding regions of defined elongation;
wherein the coating according to step (F′) is carried out by recessing the sections determined in the steps (C′) and (E′).

11. A method for selectively coating an endovascular device made of pseudoelastic alloys according to claim 10, wherein the steps (D′) and (E′) are carried out after step (A′) and before the steps (B′) and (C′).

12. A method for selectively coating an endovascular device made of pseudoelastic alloys according to claim 6, wherein the device is placed in a straight tube with a corresponding inner diameter for compression to the radially nominally loaded state according to method step (B) or (B′).

13. A method for selectively coating an endovascular device made of pseudoelastic alloys according to claim 6, wherein the device is inserted into a straight tube with a corresponding inner diameter for compression to the radially maximally loaded state according to method step (D) or (D′).

14. A method for selectively coating an endovascular device made of pseudoelastic alloys according to claim 6, wherein the elongation according to steps (C) and (E) or (C′) and (E′) is at least 3%, preferably at least 1.5% and in particular at least 1% compared to the radially unloaded state.

15. A method for selectively coating an endovascular device made of pseudoelastic alloys according to claim 6, wherein the method steps (A) to (E) or (A′) to (E′) are carried out in a computer simulation, for example by means of the finite element method (FEM).

16. An endovascular device with a coating obtainable by a method according to claim 6.

17. A system comprising an endovascular device according to claim 1 and a delivery catheter.
